(19) United States
(12) Patent Application Publication
(10) Pub. No.: US 2008/0206181 A1
(43) Pub. Date: Aug. 28, 2008

(54) Hair treatment compositions containing sophora alkaloids

(75) Inventors: QunHua Cao, Shanghai (CN); Ezat Khoshdel, Bebington (GB); Xiao-yi Pang, Shanghai (CN)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco Inc, d/b/a UNILEVER

(21) Appl. No.: 12/070,868

(22) Filed: Feb. 21, 2008

Related U.S. Application Data
(63) Continuation of application No. PCT/CN07/00594, filed on Feb. 22, 2007.

(51) Int. Cl. A61K 8/30; A61K 8/18; A61Q 5/02; A61Q 5/12

(52) U.S. Cl. 424/70.22; 424/70.1; 424/70.27

(57) ABSTRACT

The invention provides a hair treatment composition comprising one or more alkaloids derivable from plants of the Sophora genus (Sophora alkaloids), characterised in that at least 95% by weight of the total Sophora alkaloids present in the composition are tetracyclic quinolizidine alkaloids of the general structural formula (I):

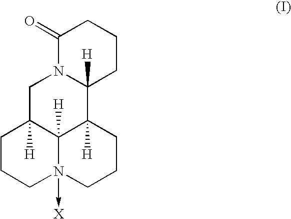

in which X is O or a lone pair.

The invention also provides the use of the above composition for the treatment of the hair fibre, especially hair fibres which are damaged.

### FIELD OF THE INVENTION

The invention relates to hair treatment compositions which comprise alkaloids derivable from plants of the Sophora genus (Sophora alkaloids). The compositions are particularly suitable for the treatment of the hair fibre.

### BACKGROUND AND PRIOR ART

Sophora, a genus of the Leguminosae family, contains several species which are important sources of traditional Chinese medicines. For example, the roots of Sophora flavescens Ait. (Chinese name “Kushen”), the roots of Sophora tonkinensis(Chinese name “Shandougen”) and the seeds of Sophora alopecuroides (Chinese name “Kudouzi”) are used in traditional Chinese medicines for the treatment of eczema, colpitis, acute pharyngolaryngeal infection, sore throat, acute dysentery and gastrointestinal haemorrhage.

Pharmacological studies have shown that the principal bioactive constituents of these traditional medicines are the quinolizidine alkaloids. Accordingly, methods have been developed for the quantitative analysis of these materials in Sophora-derived herbal medicines.

For example, Y. Yu et al./Analytica Chimica Acta 523 (2004) 15-20 report a capillary electrophoresis method permitting the simultaneous separation of seven quinolizidine alkaloids (cytisine, sophocarpine, matrine, lehmannine, sophoranol, oxymatrine and oxysophocarpine) in three kinds of Sophora medicinal plants including S. flavescens, S. tonkinensis and S. alopecuroides.

X. Chen et al./J. Chromatogr. B 812 (2004) 149-163 provides an overview of the analytical approaches used for the separation and determination of matrine-type alkaloids from S. flavescens root. The report describes the chemical structures of some of the main alkaloids identified in S. flavescens root: (−)-14β-hydroxysophoridine, (−)-sophoridine, (−)-sophoramine, (−)-12β-hydroxysophocarpine, (−)-9α-hydroxysophocarpine, (−)-sophocarpine, (+)-sophocarpine N-oxide (oxysophocarpine), (+)-matrine, (+)-matrine N-oxide (oxymatrine), (−)-14β-hydroxymatrine, (+)-9α-hydroxymatrine, (+)-lupanine, (−)-5,6-dehydrolupanine, (−)-cytisine and anagyrine. The relative content of the various alkaloids is dependent on the particular geographical origin of the source roots, as well as the number of growing years and the method of propagation.

P.-L. Ding et al./Bioorg. Med. Chem. Lett. 16 (2006)1231-1235 reports the isolation and structure elucidation of a new alkaloid, (+)-12α-hydroxysophocarpine, from the roots of Sophora flavescens, together with 10 known quinolizidine alkaloids, (+)-oxymatrine, (+)-matrine, (+)-9α-hydroxymatrine, (+)-allomatrine, (+)-oxysophocarpine, (−)-sophocarpine, (−)-9α-hydroxysophocarpine, (+)-lehmannine (−)-13,14-dehydrosophoridine, and (−)-anagyrine.

The present inventors have found that certain specific Sophora alkaloids are effective in the treatment of the hair fibre, especially hair fibres which are damaged.

The hair fibre can suffer damage from a number of sources. For example, environmental sources of hair damage include such as exposure to UV and chlorine. Chemical sources of hair damage include treatments such as bleaching, perming and straightening, and overly frequent washing with harsh surfactant-based cleansing shampoo compositions. Mechanical sources of hair damage include excessive brushing and combing and prolonged use of heated appliances for drying and styling the hair. Damage to the hair fibre typically manifests itself in cuticle and protein loss from the hair fibre, hair fibre limpness, hair fibre brittleness and breakage and frayed or split ends. Damaged hair is particularly prone to manageability problems, resulting in symptoms such as “flyaway” hair which is difficult to style or which does not retain a style, especially under conditions such as high humidity.

Compositions according to the present invention, which comprise certain specific Sophora alkaloids, are effective in the treatment, prevention and repair of hair fibre damage. The compositions are also able to impart sensory benefits to the hair fibre, such as improved smoothness, improved alignment, reduced flyaway and improved volume. U.S. Pat. No. 7,081,258 describes a composition for promoting hair growth containing, inter alia, an extract of Sophora flavescens. The extract is used for inhibiting 5α-reductase activity. No reference is made to any utility of the constituent alkaloids in the treatment of the hair fibre.

### SUMMARY OF THE INVENTION

The present invention provides a hair treatment composition comprising one or more alkaloids derivable from plants of the Sophora genus (Sophora alkaloids), characterised in that at least 95% by weight of the total Sophora alkaloids present in the composition are tetracyclic quinolizidine alkaloids of the general structural formula (I):

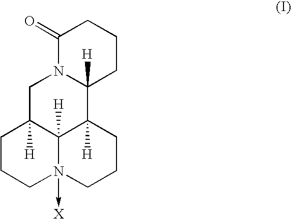

in which X is O or a lone pair.

The invention also provides the use of the above composition for the treatment of the hair fibre, especially hair fibres which are damaged.

The invention also provides a method of treating the hair fibre, especially hair fibres which are damaged, by applying the above composition to the hair.

### DETAILED DESCRIPTION AND PREFERRED EMBODIMENTS

### Sophora Alkaloids

The hair treatment composition of the invention comprises one or more Sophora alkaloids as defined above.

A tetracyclic quinolizidine alkaloid of formula (I) is which X is O is generally termed (+)-oxymatrine. The alkaloid may also be termed oxymatrine, or (+)-matrine N-oxide.

A tetracyclic quinolizidine alkaloid of formula (I) is which X is a lone pair is generally termed (+)-matrine. The alkaloid may also be termed matrine or matridin-15-one.

Preferably, at least 99% by weight of the total Sophora alkaloids present in the composition are (+)-oxymatrine and/or (+)-matrine.

More preferably the Sophora alkaloids present in the composition consist essentially of (+)-oxymatrine and/or (+)-matrine.

Most preferably the Sophora alkaloids present in the composition consist essentially of (+)-oxymatrine.

The Sophora alkaloids as described above may be obtained from their natural plant sources by extraction and purification, or alternatively they may be chemically synthesised de novo.

(+)-Oxymatrine and (+)-matrine are available commercially from suppliers such as ZiJingHua Group (P.R. China), Xi' an Tianyuan Shengwu (P.R. China) and Chemeos GmbH (Germany).

The total amount of Sophora alkaloid(s) in hair treatment compositions of the invention generally ranges from 0.001 to 10%, more preferably from 0.01 to 5%, most preferably from 0.05 to 2% (by total weight Sophora alkaloid(s) based on the total weight of the composition).

### Product Forms

Hair treatment compositions of the present invention are primarily for topical application to the hair and may be formulated as transparent or opaque emulsions, lotions, creams, pastes, sprays, mousses, waxes or gels.

Hair treatment compositions of the invention may be rinse-off products or leave-on products.

Rinse-off products are intended to be substantially rinsed off the hair of the user with water after use, such as shampoos. Rinse-off products also include conditioners which are intended for application to the hair post-wash and which may be rinsed immediately after application or (for more intensive conditioning), left on the hair for up to 2 hours, e.g. 5 minutes to 2 hours.

Leave-on products are intended not to be rinsed off the hair of the user immediately after use (i.e. within at least the first 2 hours, preferably at least four hours, after application of the product). Leave-on products include for example, lotions, creams, hair oils and leave-on conditioners for application to the hair post-wash.

Preferred product forms are shampoos, conditioners (leave-on and rinse-off) and leave-on products such as hair oils, creams and lotions.

### Shampoos

Shampoo compositions of the invention are generally aqueous, i.e. they have water or an aqueous solution or a lyotropic liquid crystalline phase as their major component.

Suitably, the shampoo composition will comprise from 50 to 98%, preferably from 60 to 90% water by weight based on the total weight of the composition.

Shampoo compositions according to the invention will typically comprise one or more anionic cleansing surfactants which are cosmetically acceptable and suitable for topical application to the hair.

### Anionic Cleansing Surfactant

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

Mixtures of any of the foregoing anionic cleansing surfactants may also be suitable.

The total amount of anionic cleansing surfactant in shampoo compositions of the invention is generally from 5 to 30, preferably from 6 to 20, more preferably from 8 to 16% by total weight anionic cleansing surfactant based on the total weight of the composition.

### Co-Surfactant

Shampoo compositions according to the invention can optionally include co-surfactants, to help impart aesthetic, physical or cleansing properties to the composition.

A preferred example is an amphoteric or zwitterionic surfactant, which can be included in an amount ranging from 0 to about 8, preferably from 1 to 4% by weight of the composition.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred example is a nonionic surfactant, which can be included in an amount ranging from 0 to 8, preferably from 2 to 5% by weight of the composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionic surfactants include mono- or di-alkyl alkanolamides. Examples include coco mono- or di-ethanolamide and coco mono-isopropanolamide.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₈ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

A preferred blend of cleansing surfactants is a combination of ammonium lauryl ether sulphate, ammonium lauryl sulphate, PEG 5 cocamide and cocamide MEA (CTFA designations).

The shampoo composition can also optionally include one or more cationic co-surfactants included in an amount ranging from 0.01 to 10, more preferably from 0.05 to 5, most preferably from 0.05 to 2% by weight of the composition. Useful cationic surfactants are described below in relation to conditioner compositions.

The total amount of surfactant (including any co-surfactant, and/or any emulsifier) in shampoo compositions of the invention is generally from 5 to 50, preferably from 5 to 30, more preferably from 10 to 25% by weight of the composition.

### Cationic Polymer

A cationic polymer is a preferred ingredient in shampoo compositions according to the invention, for enhancing conditioning performance of the shampoo.

The cationic polymer may be a homopolymer or be formed from two or more types of monomers. The molecular weight of the polymer will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic polymers can comprise mixtures of monomer units derived from amine- and/or quaternary ammonium-substituted monomer and/or compatible spacer monomers.

Suitable cationic polymers include, for example:
copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT FC 370);
copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the GAFQUAT tradename (e.g., GAFQUAT 755N);
cationic diallyl quaternary ammonium-containing polymers including, for example, dimethyldiallyammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo- and co-polymers of unsaturated carboxylic acids having from 3 to 5 carbon atoms, (as described in U.S. Pat. No. 4,009,256);
cationic polyacrylamides (as described in WO95/22311).

Other cationic polymers that can be used include cationic polysaccharide polymers, such as cationic cellulose derivatives, cationic starch derivatives, and cationic guar gum derivatives. Suitably, such cationic polysaccharide polymers have a charge density in the range from 0.1 to 4 meq/g.

Cationic polysaccharide polymers suitable for use in compositions of the invention include those of the formula:

A-O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic polymer is selected from cationic cellulose and cationic guar derivatives. Particularly preferred cationic polymers are JAGUAR C13S, JAGUAR C15, JAGUAR C17 and JAGUAR C16 and JAGUAR C162.

The cationic polymer will generally be present in compositions of the invention at levels of from 0.01 to 5, preferably from 0.05 to 1, more preferably from 0.08 to 0.5% by weight of the composition.

### Conditioner Compositions

Conditioner compositions will typically comprise one or more cationic conditioning surfactants which are cosmetically acceptable and suitable for topical application to the hair. Preferably, the cationic conditioning surfactants have the formula N⁺(R¹)(R²)(R³)(R⁴) wherein R¹, R², R³ and R⁴ are independently (C₁ to C₃₀) alkyl or benzyl.

Preferably, one, two or three of R¹, R², R³ and R⁴ are independently (C₄ to C₃₀) alkyl and the other R¹, R², R³ and R⁴ group or groups are (C₁-C₆) alkyl or benzyl.

More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (e.g., oleyl). Alkyl groups are optionally ethoxylated on the alkyl chain with one or more ethyleneoxy groups.

Suitable cationic conditioning surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (e.g., Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Another example of a class of suitable cationic conditioning surfactants for use in the invention, either alone or in admixture with one or more other cationic conditioning surfactants, is a combination of (i) and (ii) below:
(i) an amidoamine corresponding to the general formula R¹CONH(CH₂)ₘN(R²)(R³), in which R¹ is a hydrocarbyl chain having 10 or more carbon atoms, R² and R³ are independently selected from hydrocarbyl chains of from 1 to 10 carbon atoms, and m is an integer from 1 to about 10; and
(ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms,
R² and R³ are each independently hydrocarbyl residues, preferably alkyl groups, having from 1 to about 4 carbon atoms, and
m is an integer from 1 to about 4.

Preferably, R² and R³ are methyl or ethyl groups.

Preferably, m is 2 or 3, i.e. an ethylene or propylene group.

Preferred amidoamines useful herein include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylmine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

Acid (ii) may be any organic or mineral acid which is capable of protonating the amidoamine in the hair treatment composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate all the amidoamine present, i.e. at a level which is at least equimolar to the amount of amidoamine present in the composition.

In the conditioners of the invention, the level of cationic conditioning surfactant is suitably from 0.01 to 10, preferably from 0.05 to 5, more preferably from 0.1 to 2% by weight of the total composition.

### Fatty Materials

Conditioner compositions according to the invention preferably additionally comprise fatty materials.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Preferred fatty materials include cetyl alcohol, stearyl alcohol and mixtures thereof.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty material in conditioners of the invention is suitably from 0.01 to 15, preferably from 0.1 to 10, and more preferably from 0.1 to 5% by weight of the composition. The weight ratio of cationic surfactant to fatty material is suitably from 10:1 to 1:10, preferably from 4:1 to 1:8, optimally from 1:1 to 1:7, for example 1:3.

Conditioner compositions of the invention can also contain a cationic polymer. Suitable cationic polymers are described hereinabove in relation to shampoo compositions.

### Hair Oils, Creams and Lotions

Compositions of the invention may suitably take the form of a hair oil, for pre-wash or post-wash use. Typically, hair oils will predominantly comprise water-insoluble oily conditioning materials, such as triglycerides, mineral oil and mixtures thereof.

Compositions of the invention may also take the form of a hair cream or hair lotion, typically for use in between washes. Creams and lotions are aqueous emulsions comprising water-insoluble oily conditioning materials. Suitable thickeners can be included in hair creams to provides the required product viscosity. Suitable surfactants can be included in lotions to improve their stability to phase separation.

### Other Optional Ingredients

Compositions of this invention may contain any other ingredient normally used in hair treatment formulations.

### Suspending Agents

Hair treatment compositions according to the invention such as shampoos suitably comprise from 0.1 to 5% by weight of a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives. Polyacrylic acid is available commercially as Carbopol 420, Carbopol 488 or Carbopol 493. Polymers of acrylic acid cross-linked with a polyfunctional agent may also be used, they are available commercially as Carbopol 910, Carbopol 934, Carbopol 940, Carbopol 941 and Carbopol 980. An example of a suitable copolymer of a carboxylic acid containing a monomer and acrylic acid esters is Carbopol 1342. All Carbopol (trade mark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

### Further Conditioning Agents

Hair treatment compositions according to the invention such as shampoos and conditioners suitably contain further conditioning agents such as silicone conditioning agents and non-silicone oily conditioning agents.

Suitable silicone conditioning agents include polydiorganosiloxanes, in particular polydimethylsiloxanes which have the CTFA designation dimethicone. Also suitable for use in compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188. These materials can impart body, volume and stylability to hair, as well as good wet and dry conditioning. Also suitable are functionalised silicones, particularly amino-functionalised silicones.

Suitable non-silicone oily conditioning agents are selected from hydrocarbon oils, fatty esters and mixtures thereof.

The further conditioning agent is suitably present in shampoo or conditioner compositions at a level of from 0.05 to 10, preferably from 0.2 to 5, more preferably from about 0.5 to 3% by total weight of further conditioning agent based on total weight of the composition.

Hair treatment compositions of the invention may contain other optional ingredients for enhancing performance and/or consumer acceptability. Such ingredients include fragrance, dyes and pigments, pH adjusting agents, pearlescers or opacifiers, viscosity modifiers, and preservatives or antimicrobials. Each of these ingredients will be present in an amount effective to accomplish its purpose. Generally these optional ingredients are included individually at a level of up to 5% by weight of the total composition.

The invention will be further illustrated by the following, non-limiting Examples, in which all percentages quoted are by weight based on total weight unless otherwise stated.

### EXAMPLES

Matrine and oxymatrine (>96% by NMR) were purchased from Xi' an Tianyuan Shengwu.

These materials were evaluated for their ability to treat damaged hair fibres in the following tests:

### Protein Leaching

Protein leaching assays were conducted in order to find out the protective effect of test materials on protein leaching from hair.

Chinese hair fibres from the same head were washed with shampoo and air dried. Then they were immersed in ether for 2 min to remove lipid components and rinsed with running water and air. The hair fibres were cut into ˜2 mm length with scissors.

A 200 μL solution consisting of 1% test material (matrine or oxymatrine respectively) and 2% sodium dodecyl sulphate (SDS), where pH was adjusted to about 6.0 with citric acid, was incubated with 10 mg hair fibres at 37° C. for 24 h.

Protein leaching from the treated hair fibres was determined with a BCA protein assay kit.

The data were collected and analysed with One-Way ANOVA by using SPSS software. LSD was used when variances were equal, and Tamhane's T2 was used when variances were not equal.

The results are presented in Table 1 below.

TABLE 1

| | Protein leaching from hair fibres | | |
|---|---|---|---|
| | (μg/mg) | | |
| Group | Number | Average | Standard deviation |

1st Run

| | | | |
|---|---|---|---|
| Water | 5 | 0.748** | 0.033 |
| 2% SDS | 5 | 1.114 | 0.049 |
| Matrine + 2% | 5 | 0.792** | 0.034 |
| SDS | | | |
| Oxymatrine + 2% | 5 | 0.854** | 0.064 |
| SDS | | | |

2nd Run

| | | | |
|---|---|---|---|
| Water | 4 | 0.725** | 0.033 |
| 2% SDS | 4 | 1.159 | 0.048 |
| Matrine + 2% | 4 | 1.105* | 0.020 |
| SDS | | | |
| Oxymatrine + 2% | 4 | 1.035** | 0.039 |
| SDS | | | |
| Citric | 4 | 1.121 | 0.016 |
| acid + 2% SDS | | | |

Note:
All compared with 2% SDS control group,
*p < 0.05,
**p < 0.01

The results presented in Table 1 show that matrine and oxymatrine reduce protein leaching induced by a harsh surfactant such as SDS and hence reduce the fibre damage.

### Differential Scanning Calorimetry (DSC)

To prepare damaged hair fibre, European hair fibres were bleached with L' Oreal Platifiz precision powder and Oxydant crème (1:1.5) for 30 min, rinsed completely with running tap water and naturally dried overnight before the next application. These hair fibres were immersed in a 1% aqueous solution of test material (matrine or oxymatrine respectively) and water (as a control) at pH 5.5 (adjusted with HCl and NaOH) for 1 hour respectively, rinsed with distilled water for 30 sec and naturally dried overnight and then they were cut into ˜2 mm lengths with scissors.

For the DSC investigation a Mettler Toledo DSC823e analyser was used. About 6 mg of sample was weighed into a pressure resistant (20 bar), stainless steel, large volume pan (120 μl capacity). 50 μl of water was added and the pan was sealed. Samples were then mixed using a rotary mixer and left overnight to allow the water to equilibrate throughout the sample. Samples were run through a temperature programme of 40 to 80° C. at a rate of 10° C./min in 30 ml/min nitrogen atmosphere. The helix transition temperature was collected and analyzed with one-way ANOVA. Each sample was carried out three times.

The results are presented in Table 2 below.

TABLE 2

| | Average | | | |
|---|---|---|---|---|
| | Denaturation | | Sub | p value |
| | Temperature | | From | (One way |
| Hair | Td (° C.) | St. Dev. | damaged | ANOVA) |
| 1st run | | | | |
| Virgin | 152.05** | 0.41 | 9.48 | 0.000 |
| Damaged | 142.57 | 0.63 | | \ |
| Matrine | 148.00** | 0.16 | 5.43 | 0.000 |
| Oxymatrine | 152.24** | 0.07 | 9.68 | 0.000 |
| 2nd run | | | | |
| Virgin | 153.19** | 0.58 | 10.01 | 0.000 |
| Damaged | 143.18 | 0.25 | | \ |
| Matrine | 148.07** | 0.38 | 4.89 | 0.000 |
| Oxymatrine | 150.67** | 0.22 | 7.49 | 0.000 |

Note
*p < 0.05 and **p < 0.01 compared with damaged hair

The results presented in Table 2 show that matrine and oxymatrine enhance the denaturation temperature of damaged hair fibre by 5.43 and 9.68° C. respectively. The denaturation temperature of damaged hair fibre treated with matrine (148° C.) and oxymatrine (152.24° C.) approaches that of virgin hair (152.05° C.). This implies that matrine and particularly oxymatrine fully repair the damage caused by bleaching hair fibre.

### Sensory Evaluation

Matrine and oxymatrine were each formulated at 1% in a shampoo base. The control was the shampoo base without either matrine or oxymatrine. The shampoos were assessed by an expert stylist. The assessment was a half-head mannequin direct comparison of the control vs. the test formulation after styling.

The half-head mannequin evaluation results are presented in Table 3 below.

TABLE 3

| | Wins for | |
|---|---|---|
| | oxymatrine | Wins for |
| Attribute | formulation | control |
| Lack of flyaway | 5 | 2 |
| Smoothness | 5 | 2 |
| Less fluffy | 6 | 1 |
| Alignment | 7 | 0 |
| | Wins for | |
| | matrine | Wins for |
| Attribute | formulation | control |
| Lack of flyaway | 5 | 1 |
| Smoothness | 4 | 2 |
| Less fluffy | 4 | 2 |
| Alignment | 4 | 2 |

The results presented in Table 3 show that shampoo formulations incorporating either matrine or oxymatrine impart superior sensory benefits to the hair fibre when compared to the control shampoo with neither material.

Additional benefits observed by the expert stylist for the shampoo formulations incorporating either matrine or oxymatrine (compared to the control) included improved damage repair, ease of blow dry and improved hair volume creation in the absence of added styling products.

The following Examples 1 and 2 illustrate shampoo compositions according to the invention:

| | Example 1 | Example 2 |
|---|---|---|
| Sodium lauryl ether sulphate | 12 | 12 |
| (2EO) | | |
| Cocoylamidopropyldimethyl | 2 | 2 |
| glycine | | |
| Silicone emulsion | 2 | 2 |
| Guar hydroxypropyl | 0.30 | 0.30 |
| trimethylammonium chloride | | |
| Preservative | 0.35 | 0.35 |
| Perfume | 0.42 | 0.42 |
| Citric acid | 0.17 | 0.17 |
| Matrine | — | 1.0 |
| Oxymatrine | 1.0 | — |
| Water and Minors | To 100 | |
| | Weight % | |

1. A hair treatment composition comprising one or more alkaloids derivable from plants of the Sophora genus (Sophora alkaloids), characterised in that at least 95% by weight of the total Sophora alkaloids present in the composition are tetracyclic quinolizidine alkaloids of the general structural formula (I): 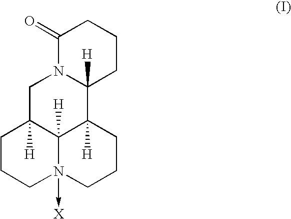
in which X is O or a lone pair.

2. A composition according to claim 1, in which at least 99% by weight of the total Sophora alkaloids present in the composition are (+)-oxymatrine and/or (+)-matrine.

3. A composition according to claim 2, in which the Sophora alkaloids present in the composition consist essentially of (+)-oxymatrine and/or (+)-matrine.

4. A composition according to claim 1, characterised in that it is a shampoo composition comprising at one or more anionic cleansing surfactants in an amount of from 5 to 30% by total weight anionic cleansing surfactant based on the total weight of the composition.

5. A composition according to claim 1 characterised in that it is a conditioner composition comprising one or more cationic conditioning surfactants in an amount of from 0.01 to 10% by total weight cationic conditioning surfactant based on the total weight of the composition.

6. A composition according to claim 1 characterised in that it is a hair oil, cream or lotion.

7. A composition according to claim 1 characterised in that the amount of Sophora alkaloid(s) ranges from 0.001 to 10%, more preferably from 0.01 to 5%, most preferably from 0.05 to 2% (by total weight Sophora alkaloid(s) based on the total weight of the composition).

8. A method of treating the hair fibre, especially hair fibres which are damaged, which method comprises topically applying a composition according to claim 1 to the hair.

9. The use of a composition according to claim 1 for the treatment of the hair fibre, especially hair fibres which are damaged.
